(19) United States
(12) Patent Application Publication
(10) Pub. No.: US 2019/0341154 A1
(43) Pub. Date: Nov. 7, 2019

(54) Dynamically Generating Patient-Facing Mobile Interfaces

(71) Applicant: Liquid Payments, Inc., Fort Lauderdale, FL (US)

(72) Inventor: Shashi B. Kapur, Fort Lauderdale, FL (US)

(21) Appl. No.: 16/402,773

(22) Filed: May 3, 2019

Related U.S. Application Data
(60) Provisional application No. 62/666,210, filed on May 3, 2018.

(51) Int. Cl. G16H 80/00; G16H 10/60; G06Q 20/10; G06Q 20/32; G06Q 20/36; G06Q 40/08

(52) U.S. Cl. CPC G16H 80/00; G16H 10/60; G06Q 40/08; G06Q 20/3223; G06Q 20/3672; G06Q 20/102

(57) ABSTRACT

Methods, systems, computer-readable media, and apparatuses for dynamically generating patient-facing mobile interfaces based on healthcare data retrieved from secure medical information systems are presented. A computing platform may receive, from a first patient mobile device, a first request for a first link comprising a first unique token. Responsive to receiving the first request for the first link comprising the first unique token, the computing platform may load one or more patient-specific records associated with the first link comprising the first unique token. Subsequently, the computing platform may generate a first mobile explanation-of-benefits user interface based on the one or more patient-specific records associated with the first link comprising the first unique token. Then, the computing platform may send, to the first patient mobile device, the first mobile explanation-of-benefits user interface generated based on the one or more patient-specific records associated with the first link comprising the first unique token.

### CROSS-REFERENCE TO RELATED APPLICATIONS

This application claims the benefit of and priority to U.S. Provisional Patent Application Ser. No. 62/666,210, filed May 3, 2018, and entitled “Enhanced Medical EOB Interfaces,” which is incorporated by reference herein in its entirety.

### BACKGROUND

Aspects of the disclosure relate to data processing, preventing unauthorized access to secure information systems, and securely providing access to healthcare information. In particular, one or more aspects of the disclosure relate to dynamically generating patient-facing mobile interfaces based on healthcare data retrieved from secure medical information systems.

Generating, tracking, controlling, and otherwise handling computer-processed events that affect multiple computer systems may present a number of technical challenges, such as ensuring loss-less data transfers across different computer systems while also optimizing for both processing power consumption and network bandwidth usage throughout the associated computing infrastructure. In many instances, these challenges are compounded when computer systems are handling private and confidential data, such as medical-related information, because maintaining the security and integrity of such data may be critically important to the underlying applications and achieving these goals may require using additional computing resources.

### SUMMARY

Aspects of the disclosure provide effective, efficient, scalable, and convenient technical solutions that address and overcome the technical problems associated with handling computer-processed events that affect multiple computer systems. In particular, one or more aspects of the disclosure relate to technical solutions for dynamically generating patient-facing mobile interfaces based on healthcare data retrieved from secure medical information systems.

In accordance with one or more embodiments, a computing platform having at least one processor, a communication interface, and memory may receive, via the communication interface, from a first patient mobile device, a first request for a first link comprising a first unique token. Responsive to receiving the first request for the first link comprising the first unique token, the computing platform may load one or more patient-specific records associated with the first link comprising the first unique token. Subsequently, the computing platform may generate a first mobile explanation-of-benefits user interface based on the one or more patient-specific records associated with the first link comprising the first unique token. Then, the computing platform may send, via the communication interface, to the first patient mobile device, the first mobile explanation-of-benefits user interface generated based on the one or more patient-specific records associated with the first link comprising the first unique token.

In some embodiments, prior to receiving the first request for the first link comprising the first unique token, the computing platform may receive, via the communication interface, from a first healthcare provider computer system, a first data file comprising at least one patient-specific record associated with at least one patient. Subsequently, the computing platform may parse the first data file received from the first healthcare provider computer system. Based on parsing the first data file received from the first healthcare provider computer system, the computing platform may generate one or more patient-specific notifications. Then, the computing platform may send, via the communication interface, to one or more patient mobile devices, the one or more patient-specific notifications generated based on parsing the first data file received from the first healthcare provider computer system. In addition, sending the one or more patient-specific notifications generated based on parsing the first data file received from the first healthcare provider computer system to the one or more patient mobile devices may include sending a first notification to the first patient mobile device, and the first notification may include the first link comprising the first unique token.

In some embodiments, the first healthcare provider computer system may include a secure file transfer protocol (SFTP) server. In addition, receiving the first data file comprising the at least one patient-specific record associated with the at least one patient from the first healthcare provider computer system may include: establishing a secure connection with the SFTP server; and downloading the first data file comprising the at least one patient-specific record associated with the at least one patient via the secure connection with the SFTP server.

In some embodiments, generating the first mobile explanation-of-benefits user interface based on the one or more patient-specific records associated with the first link comprising the first unique token may include: identifying one or more current procedural terminology (CPT) codes as being present in the one or more patient-specific records associated with the first link comprising the first unique token; translating the one or more CPT codes identified as being present in the one or more patient-specific records into one or more visit descriptions; and inserting the one or more visit descriptions into the first mobile explanation-of-benefits user interface.

In some embodiments, generating the first mobile explanation-of-benefits user interface based on the one or more patient-specific records associated with the first link comprising the first unique token may include inserting, based on the one or more patient-specific records, a physician name, a patient name, a claim-paid amount, and a payment-due amount into the first mobile explanation-of-benefits user interface.

In some embodiments, generating the first mobile explanation-of-benefits user interface based on the one or more patient-specific records associated with the first link comprising the first unique token may include inserting promotional content into the first mobile explanation-of-benefits user interface based on the one or more patient-specific records.

In some embodiments, inserting the promotional content into the first mobile explanation-of-benefits user interface based on the one or more patient-specific records may include selecting the promotional content from a library of available promotional content based on identifying one or more codes as being present in the one or more patient-specific records associated with the first link comprising the first unique token.

In some embodiments, inserting the promotional content into the first mobile explanation-of-benefits user interface based on the one or more patient-specific records may include selecting the promotional content from a library of available promotional content based on identifying a physician name included in the one or more patient-specific records associated with the first link comprising the first unique token.

In some embodiments, the computing platform may receive, via the communication interface, from the first patient mobile device, a request to submit a payment associated with a payment-due amount included in the first mobile explanation-of-benefits user interface. Responsive to receiving the request to submit the payment associated with the payment-due amount included in the first mobile explanation-of-benefits user interface, the computing platform may determine whether saved payment method information linked to a user of the first patient mobile device is available. Based on determining that the saved payment method information linked to the user of the first patient mobile device is available, the computing platform may process the payment associated with the payment-due amount included in the first mobile explanation-of-benefits user interface using the saved payment method information linked to the user of the first patient mobile device.

In some embodiments, based on determining that the saved payment method information linked to the user of the first patient mobile device is not available, the computing platform may generate a first payment-details user interface based on the one or more patient-specific records associated with the first link comprising the first unique token. Subsequently, the computing platform may send, via the communication interface, to the first patient mobile device, the first payment-details user interface generated based on the one or more patient-specific records associated with the first link comprising the first unique token.

In some embodiments, generating the first payment-details user interface based on the one or more patient-specific records associated with the first link comprising the first unique token may include inserting promotional content into the first payment-details user interface based on the one or more patient-specific records.

In some embodiments, after sending the first payment-details user interface to the first patient mobile device, the computing platform may receive, via the communication interface, from the first patient mobile device, new payment information linked to the user of the first patient mobile device. Subsequently, the computing platform may store the new payment information linked to the user of the first patient mobile device received from the first patient mobile device.

In some embodiments, after storing the new payment information linked to the user of the first patient mobile device received from the first patient mobile device, the computing platform may process the payment associated with the payment-due amount included in the first mobile explanation-of-benefits user interface using the new payment information linked to the user of the first patient mobile device.

In some embodiments, after processing the payment associated with the payment-due amount included in the first mobile explanation-of-benefits user interface, the computing platform may generate a second mobile explanation-of-benefits user interface for the user of the first patient mobile device. Subsequently, the computing platform may send, via the communication interface, to the first patient mobile device, the second mobile explanation-of-benefits user interface generated for the user of the first patient mobile device.

In some embodiments, the computing platform may receive, via the communication interface, from a second patient mobile device, a second request for a second link comprising a second unique token. Responsive to receiving the second request for the second link comprising the second unique token, the computing platform may load one or more patient-specific records associated with the second link comprising the second unique token. Subsequently, the computing platform may generate a third mobile explanation-of-benefits user interface based on the one or more patient-specific records associated with the second link comprising the second unique token. Then, the computing platform may send, via the communication interface, to the second patient mobile device, the third mobile explanation-of-benefits user interface generated based on the one or more patient-specific records associated with the second link comprising the second unique token.

These features, along with many others, are discussed in greater detail below.

### BRIEF DESCRIPTION OF THE DRAWINGS

The present disclosure is illustrated by way of example and not limited in the accompanying figures in which like reference numerals indicate similar elements and in which:

FIGS. 1A and 1B depict an illustrative computing environment for dynamically generating patient-facing mobile interfaces based on healthcare data retrieved from secure medical information systems in accordance with one or more example embodiments;

FIGS. 2A-2H depict an illustrative event sequence for dynamically generating patient-facing mobile interfaces based on healthcare data retrieved from secure medical information systems in accordance with one or more example embodiments;

FIG. 3 depicts an example dataset that may be used in dynamically generating patient-facing mobile interfaces based on healthcare data retrieved from secure medical information systems in accordance with one or more example embodiments;

FIGS. 4-7 depict example graphical user interfaces for dynamically generating patient-facing mobile interfaces based on healthcare data retrieved from secure medical information systems in accordance with one or more example embodiments; and

FIG. 8 depicts an illustrative method for dynamically generating patient-facing mobile interfaces based on healthcare data retrieved from secure medical information systems in accordance with one or more example embodiments.

### DETAILED DESCRIPTION

In the following description of various illustrative embodiments, reference is made to the accompanying drawings, which form a part hereof, and in which is shown, by way of illustration, various embodiments in which aspects of the disclosure may be practiced. It is to be understood that other embodiments may be utilized, and structural and functional modifications may be made, without departing from the scope of the present disclosure.

It is noted that various connections between elements are discussed in the following description. It is noted that these connections are general and, unless specified otherwise, may be direct or indirect, wired or wireless, and that the specification is not intended to be limiting in this respect.

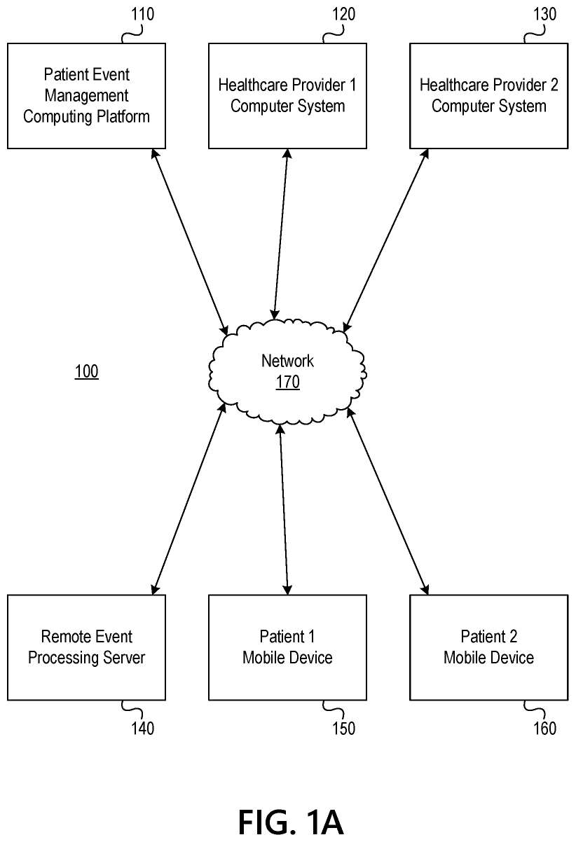

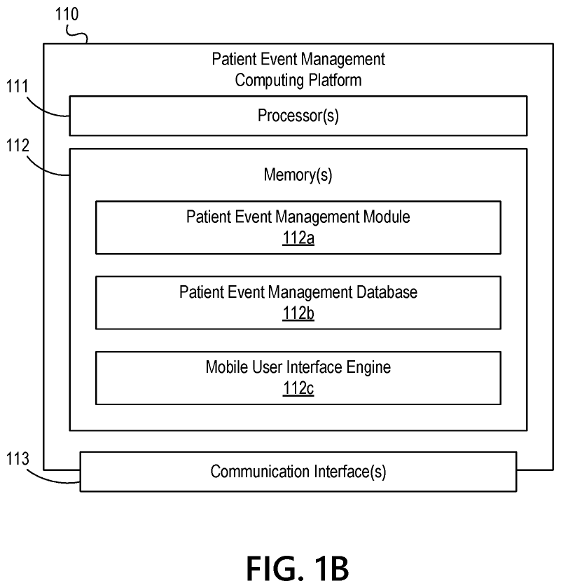

FIGS. 1A and 1B depict an illustrative computing environment for dynamically generating patient-facing mobile interfaces based on healthcare data retrieved from secure medical information systems in accordance with one or more example embodiments. Referring to FIG. 1A, computing environment 100 may include one or more computer systems. For example, computing environment 100 may include a patient event management computing platform 110, a first healthcare provider computer system 120, a second healthcare provider computer system 130, a remote event processing server 140, a first patient mobile device 150, and a second patient mobile device 160.

As illustrated in greater detail below, patient event management computing platform 110 may include one or more computing devices configured to perform one or more of the functions described herein. For example, patient event management computing platform 110 may include one or more computers (e.g., laptop computers, desktop computers, servers, server blades, or the like).

Healthcare provider computer system 120 may be a collection of networked computing devices that may include and/or function as a practice management system, an electronic medical record (EMR) system, or the like. For example, healthcare provider computer system 120 may include one or more personal computing devices (e.g., desktop computers, laptop computers) and/or mobile computing devices (e.g., smartphones, tablets, wearable devices) that may be linked to and/or used by a first healthcare provider user and/or user group (e.g., a medical practice group, a physician, a nurse, a physician office administrator) at a first healthcare provider location (e.g., a hospital, a clinic, a doctor's office). Healthcare provider computer system 130 also may be a collection of networked computing devices that may include and/or function as a practice management system, an electronic medical record (EMR) system, or the like. For example, healthcare provider computer system 130 may include one or more personal computing devices (e.g., desktop computers, laptop computers) and/or mobile computing devices (e.g., smartphones, tablets, wearable devices) that may be linked to and/or used by a second healthcare provider user and/or user group (e.g., a medical practice group, a physician, a nurse, a physician office administrator) at a second healthcare provider location (e.g., a hospital, a clinic, a doctor's office). The second healthcare provider user and/or user group may be different from the first healthcare provider user and/or user group (e.g., a different medical practice group, physician, nurse, or physician office administrator), and the second healthcare provider location may be different from the first healthcare provider location (e.g., a different hospital, clinic, or doctor's office).

Remote event processing server 140 may be a server computer system that is owned, operated, and/or maintained by a third-party payment processing provider and/or payment network. In some instances, remote event processing server 140 may expose and/or provide one or more remotely-accessible computer-executable functions, such as via a web application programming interface (API), that can be invoked and/or otherwise used by other computer systems and/or devices in computing environment 100 to request, schedule, and/or process payment transactions (e.g., credit card payment transactions, debit card payment transactions, etc.). For example, such a web application programming interface may define a set of functions and associated syntax for such functions, and other computer systems and/or devices in computing environment 100 may connect to remote event processing server 140 and, while a connection or other communications link is established to remote event processing server 140, generate and/or send API calls and/or other commands to remote event processing server 140 in accordance with the defined syntax to direct and/or otherwise cause remote event processing server 140 to execute one or more functions of the set of functions that may be provided by remote event processing server 140.

Patient mobile device 150 may a personal computing device (e.g., desktop computer, laptop computer) or mobile computing device (e.g., smartphone, tablet, wearable device) that may be linked to and/or used by a first patient user (who may, e.g., be a patient of a healthcare provider who has visited, is visiting, or will visit the first healthcare provider location or the second healthcare provider location). Patient mobile device 160 may a personal computing device (e.g., desktop computer, laptop computer) or mobile computing device (e.g., smartphone, tablet, wearable device) that may be linked to and/or used by a second patient user (who may, e.g., be a patient of a healthcare provider who has visited, is visiting, or will visit the first healthcare provider location or the second healthcare provider location). The second patient user may, for instance, be a different user (e.g., a different patient) from the first patient user.

Computing environment 100 also may include one or more networks, which may interconnect one or more of patient event management computing platform 110, healthcare provider computer system 120, healthcare provider computer system 130, remote event processing server 140, patient mobile device 150, and patient mobile device 160. For example, computing environment 100 may include a network 170, which may include one or more private networks, public networks, and/or sub-networks. In addition, network 170 may interconnect one or more of patient event management computing platform 110, healthcare provider computer system 120, healthcare provider computer system 130, remote event processing server 140, patient mobile device 150, patient mobile device 160, and one or more other computer systems and/or devices connected to the one or more private networks, public networks, and/or sub-networks included in network 170.

In one or more arrangements, healthcare provider computer system 120, healthcare provider computer system 130, remote event processing server 140, patient mobile device 150, patient mobile device 160, and/or the other systems included in computing environment 100 may be any type of computing device capable of receiving a user interface, receiving input via the user interface, establishing one or more connections to one or more other computing devices, and communicating the received input to the one or more other computing devices while the one or more connections are established. For example, healthcare provider computer system 120, healthcare provider computer system 130, remote event processing server 140, patient mobile device 150, patient mobile device 160, and/or the other systems included in computing environment 100 may, in some instances, be and/or include server computers, desktop computers, laptop computers, tablet computers, smart phones, or the like that may include one or more processors, memories, communication interfaces, storage devices, and/or other components. As noted above, and as illustrated in greater detail below, any and/or all of patient event management computing platform 110, healthcare provider computer system 120, healthcare provider computer system 130, remote event processing server 140, patient mobile device 150, and patient mobile device 160 may, in some instances, be special-purpose computing devices configured to perform specific functions.

Referring to FIG. 1B, patient event management computing platform 110 may include one or more processor(s) 111, memory(s) 112, and communication interface(s) 113. A data bus may interconnect processor(s) 111, memory(s) 112, and communication interface(s) 113. Communication interface(s) 113 may be one or more network interfaces configured to support communication between patient event management computing platform 110 and one or more networks (e.g., network 170). Memory(s) 112 may include one or more program modules having instructions that when executed by processor(s) 111 cause patient event management computing platform 110 to perform one or more functions described herein and/or one or more databases that may store and/or otherwise maintain information which may be used by such program modules and/or processor(s) 111. In some instances, the one or more program modules and/or databases may be stored by and/or maintained in different memory units of patient event management computing platform 110 and/or by different computing devices that may form and/or otherwise make up patient event management computing platform 110. In addition, memory(s) 112 may have, store, and/or include a patient event management module 112a, a patient event management database 112b, and a mobile user interface engine 112c. Patent event management module 112a may have, store, and/or include instructions that, when executed, direct and/or cause patient event management computing platform 110 to dynamically generate patient-facing mobile interfaces based on healthcare data retrieved from secure medical information systems and/or cause patient event management computing platform 110 to perform other functions, as discussed in greater detail below. Patient event management database 112b may store information used by patient event management module 112a and/or patient event management computing platform 110 in dynamically generating patient-facing mobile interfaces based on healthcare data retrieved from secure medical information systems and/or in performing other functions. Mobile user interface engine 112c may have, store, and/or include user interface templates, instructions, and/or other data that enable and/or cause patient event management computing platform 110 to generate user interfaces and/or user interface elements associated with patient-facing mobile interfaces, as discussed in greater detail below. In one or more arrangements, patient event management module 112a, patient event management database 112b, mobile user interface engine 112c, and/or patient event management computing platform 110 may be configured to be compliant with Health Insurance Portability and Accountability Act (HIPAA), Payment Card Industry Data Security Standard (PCI DSS), and/or one or more other laws, regulations, and/or standards, to ensure the privacy, confidentiality, security, and integrity of both the medical-related data, which may include patient data, and the payments data processed by patient event management computing platform 110.

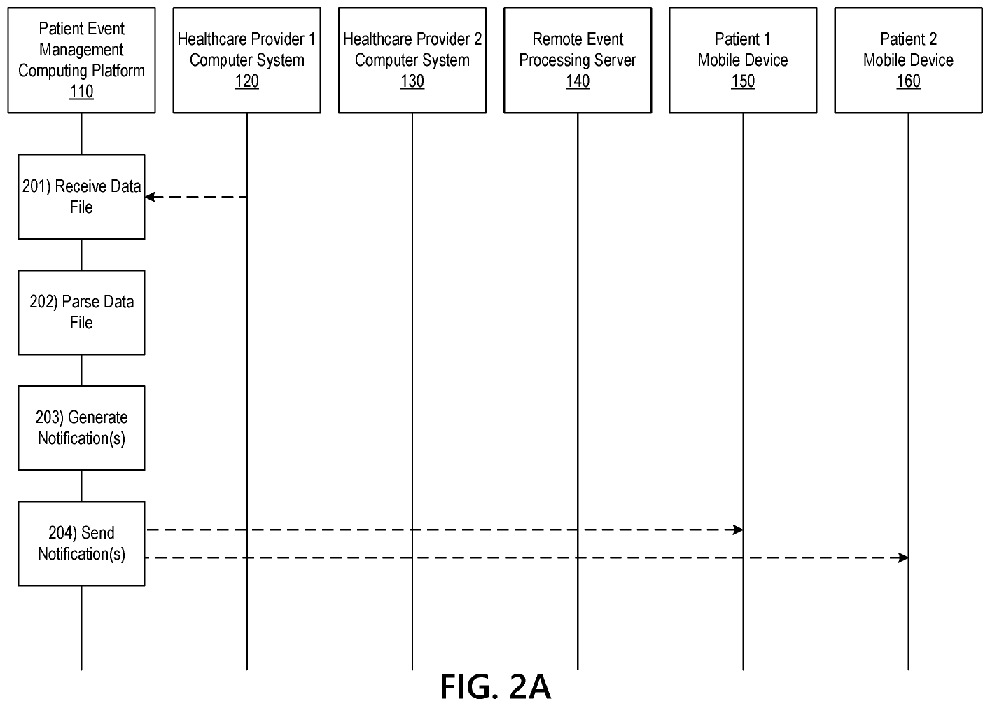

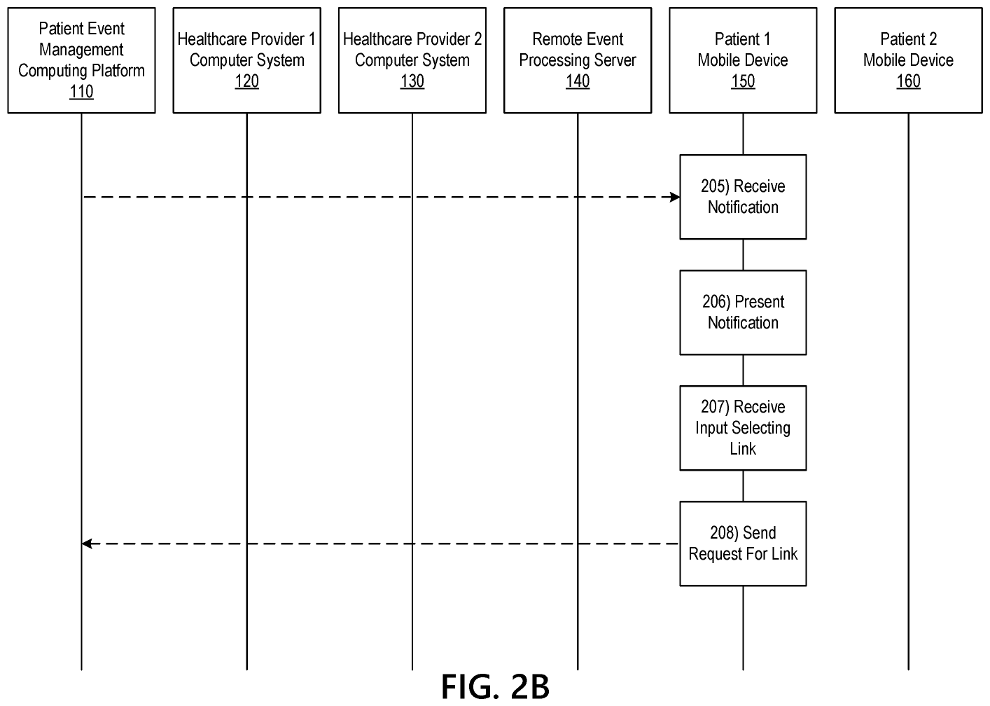

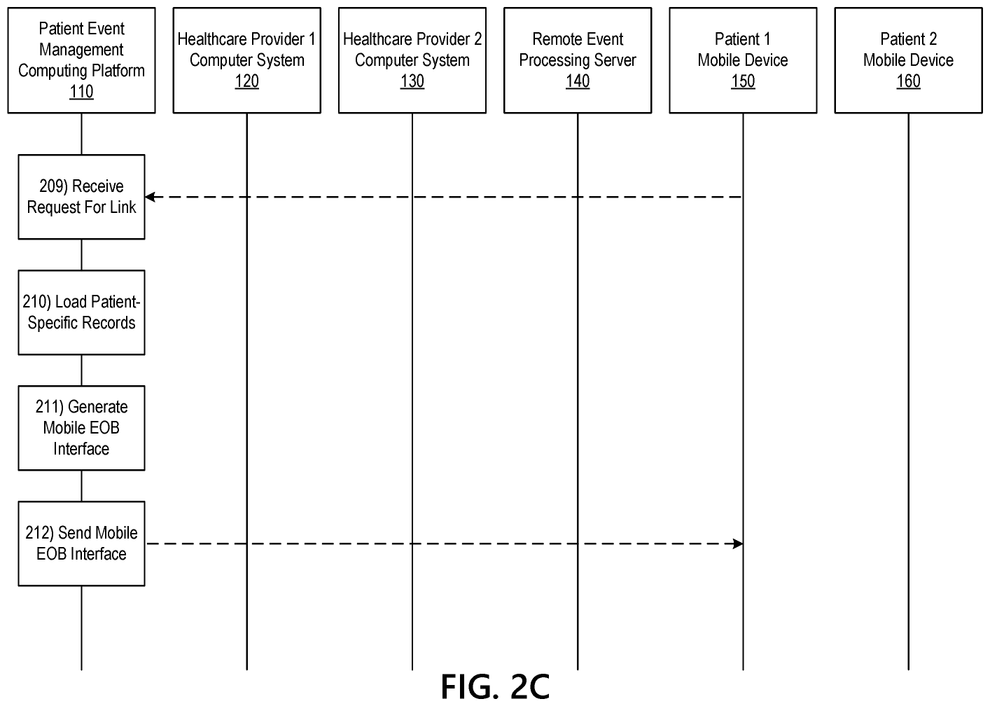

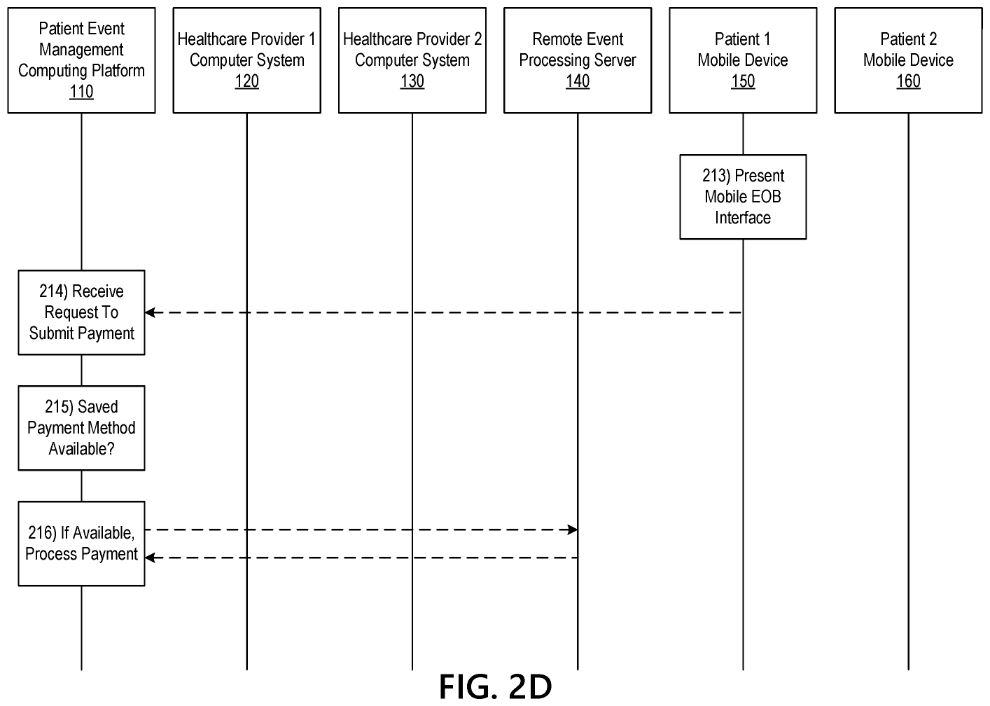

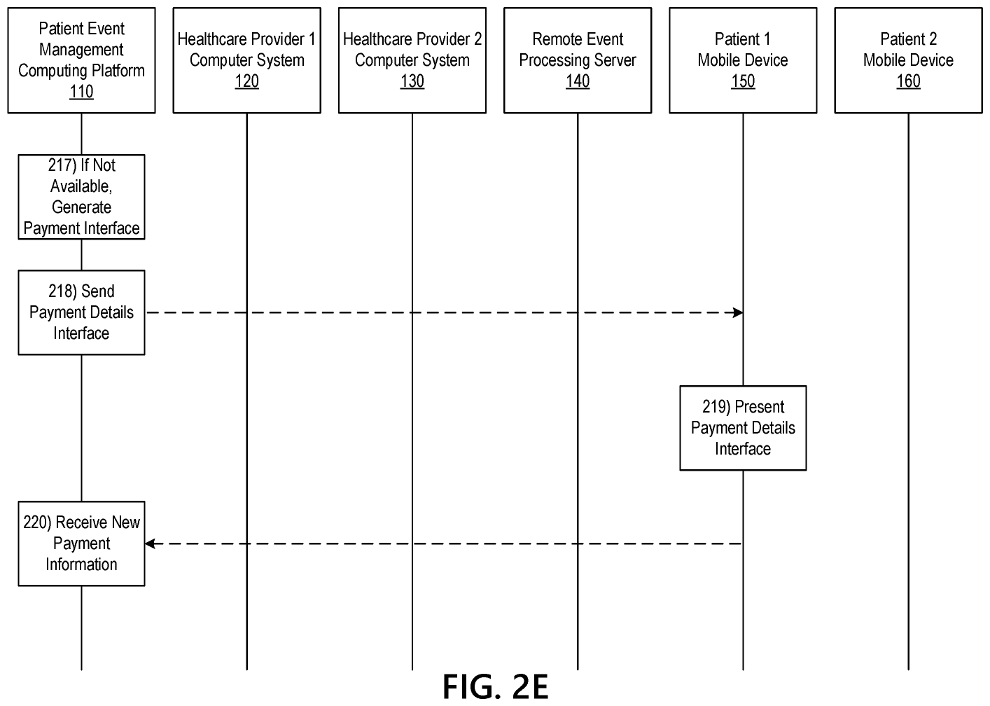

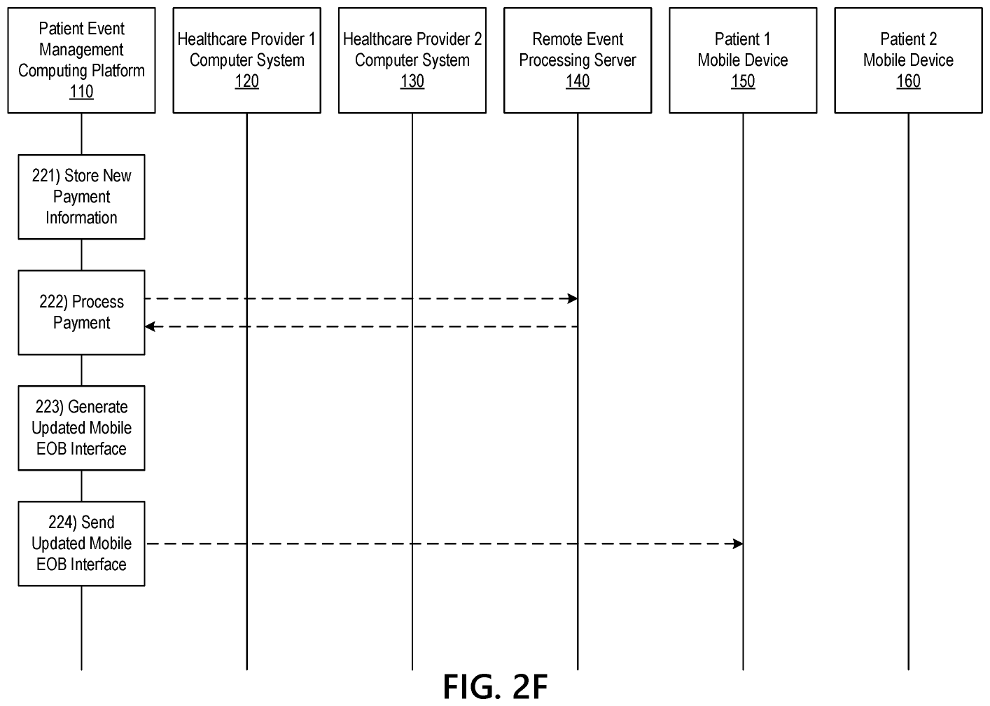

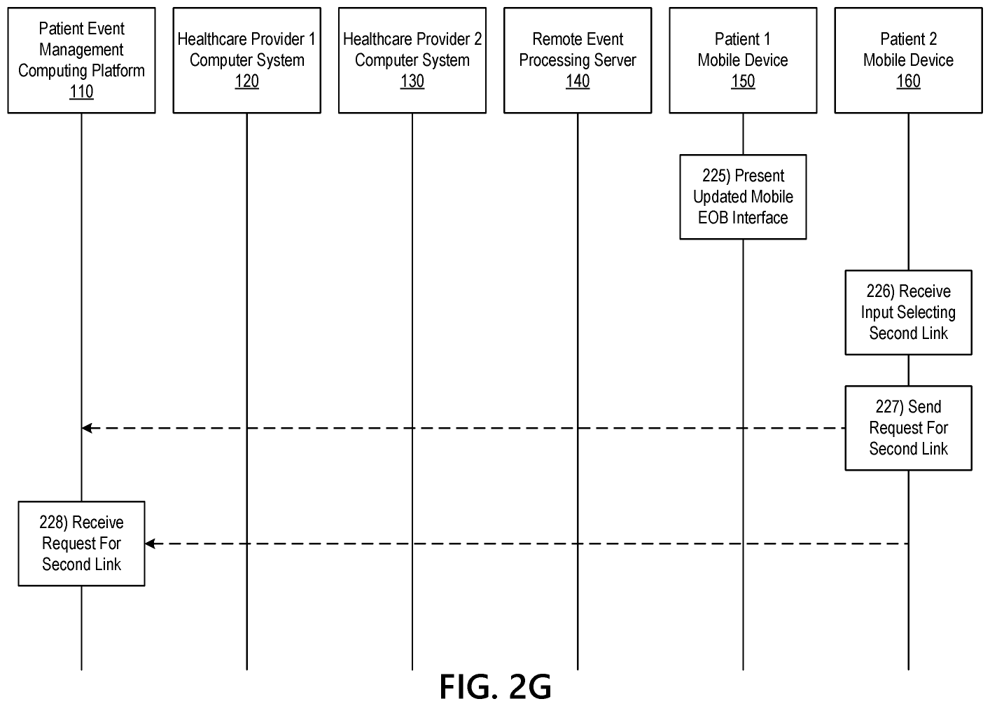

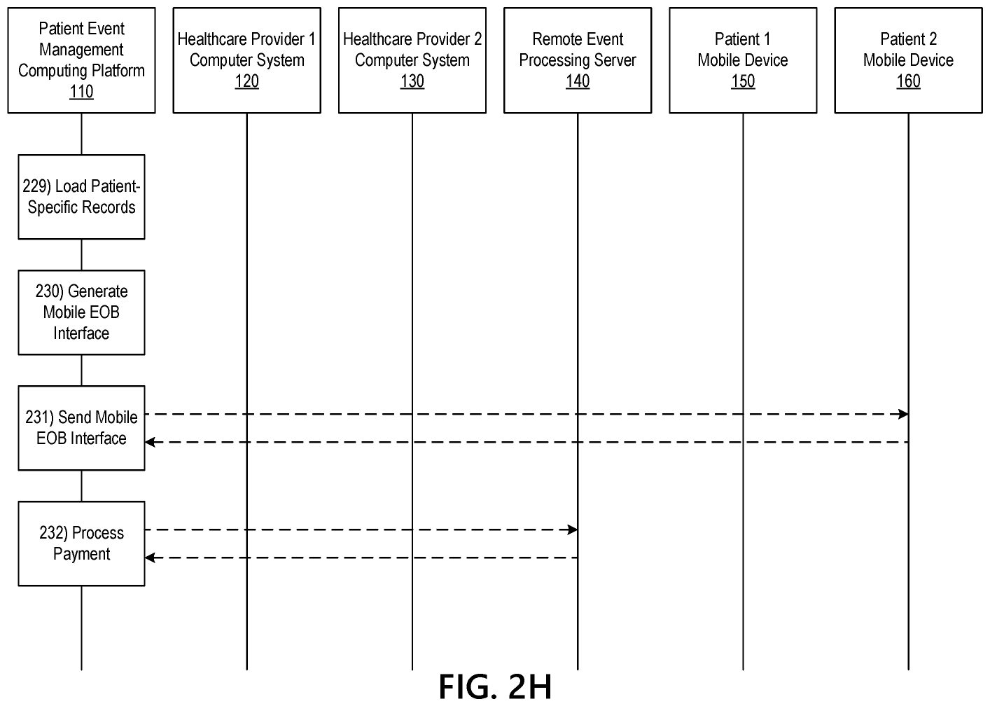

FIGS. 2A-2H depict an illustrative event sequence for dynamically generating patient-facing mobile interfaces based on healthcare data retrieved from secure medical information systems in accordance with one or more example embodiments. Referring to FIG. 2A, at step 201, patient event management computing platform 110 may receive a data file from healthcare provider computer system 120. For example, at step 201, prior to receiving a first request for a first link comprising a first unique token, patient event management computing platform 110 may receive, via the communication interface (e.g., communication interface 113), from a first healthcare provider computer system (e.g., healthcare provider computer system 120), a first data file comprising at least one patient-specific record associated with at least one patient.

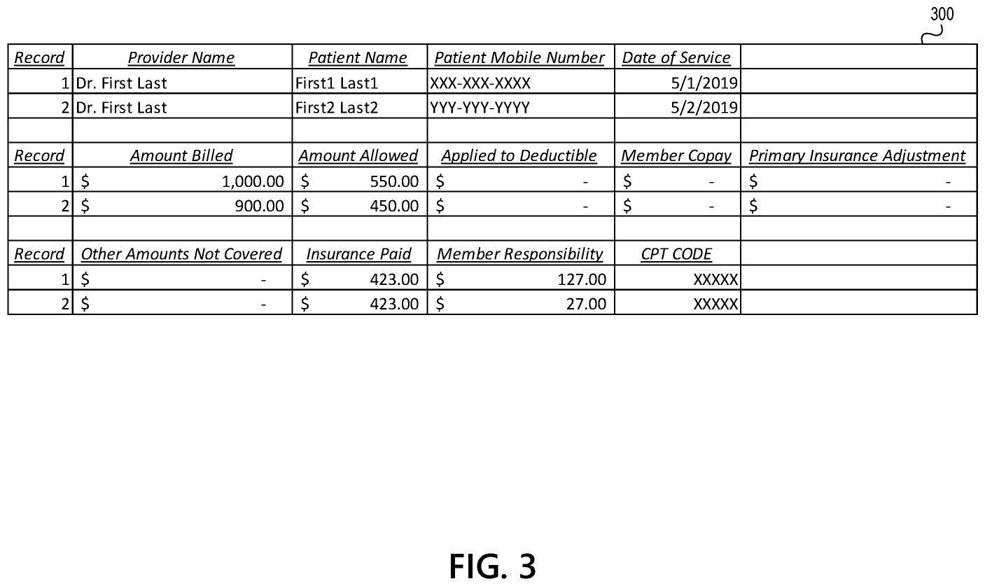

For instance, at step 201, patient event management computing platform 110 may receive a delimited text file (e.g., a comma-separated value (CSV) file), a spreadsheet file, or another type of data file that may include explanation-of-benefits information associated with various patients of a practice group or physician associated with healthcare provider computer system 120. In some instances, healthcare provider computer system 120 may include a practice management system and/or an EMR system that may be configured to periodically run custom reports, generate such delimited text files, and send the delimited text files to patient event management computing platform 110 (e.g., to facilitate patient payments). In some instances, the data file (which may, e.g., be received by patient event management computing platform 110 at step 201) may include a dataset similar to example dataset 300, which is illustrated in FIG. 3. As seen in FIG. 3, dataset 300 may include a plurality of records associated with a plurality of patients. For each record, dataset 300 may include fields and/or values identifying a provider name, a patient name, a patient mobile telephone number, a date of service, an amount billed, an amount allowed, an applied to deductible amount, a member copay amount, a primary insurance adjustment amount, an other-amounts-not-covered value, an insurance paid amount, a member responsibility amount, and a current procedural terminology (CPT) code. As illustrated below, any and/or all of the information included in such a dataset (e.g., dataset 300) may be used by patient event management computing platform 110 in generating a mobile explanation-of-benefits user interface for a patient associated with a particular record included in the dataset.

In some embodiments, the first healthcare provider computer system may include a secure file transfer protocol (SFTP) server. In addition, receiving the first data file comprising the at least one patient-specific record associated with the at least one patient from the first healthcare provider computer system may include: establishing a secure connection with the SFTP server; and downloading the first data file comprising the at least one patient-specific record associated with the at least one patient via the secure connection with the SFTP server. For example, in some instances, healthcare provider computer system 120 may include a secure file transfer protocol (SFTP) server. In addition, in receiving the first data file comprising the at least one patient-specific record associated with the at least one patient from the first healthcare provider computer system, patient event management computing platform 110 may establish a secure connection with the SFTP server (which may, e.g., be included in healthcare provider computer system 120). Then, patient event management computing platform 110 may download the first data file comprising the at least one patient-specific record associated with the at least one patient via the secure connection with the SFTP server (which may, e.g., be included in healthcare provider computer system 120).

At step 202, patient event management computing platform 110 may parse the data file received at step 201. For example, at step 202, patient event management computing platform 110 may parse the first data file received from the first healthcare provider computer system (e.g., healthcare provider computer system 120). In some instances, patient event management computing platform 110 may parse the data file using one or more regular expressions, delimiters, and/or the like, so as to identify the data elements and/or values associated with different records and/or fields included in the data file.

At step 203, patient event management computing platform 110 may generate one or more notifications for one or more patients and/or patient devices. For example, at step 203, based on parsing the first data file received from the first healthcare provider computer system (e.g., healthcare provider computer system 120), patient event management computing platform 110 may generate one or more patient-specific notifications. For instance, patient event management computing platform 110 may generate a notification for each record included in the data file, and the notification may be directed to a patient identified in the record based on the patient's name, phone number, and/or other details included in the record. In addition, patient event management computing platform 110 may generate a unique token that corresponds to each notification and record. Each unique token may be an alphanumeric string that is randomly generated by patient event management computing platform 110. In addition, by associating each notification and record with a unique token, patient event management computing platform 110 may enable a patient-user to quickly and easily access their own explanation-of-benefits record(s) by requesting the token(s) corresponding to the notification(s) received by the particular patient-user.

At step 204, patient event management computing platform 110 may send the one or more notifications to one or more patients and/or patient devices. For example, at step 204, patient event management computing platform 110 may send, via the communication interface (e.g., communication interface 113), to one or more patient mobile devices (e.g., patient mobile device 150, patient mobile device 160), the one or more patient-specific notifications generated based on parsing the first data file received from the first healthcare provider computer system (e.g., healthcare provider computer system 120). In some embodiments, sending the one or more patient-specific notifications generated based on parsing the first data file received from the first healthcare provider computer system to the one or more patient mobile devices may include sending a first notification to a first patient mobile device, and the first notification may include a first link comprising a first unique token. For example, in sending the one or more patient-specific notifications generated based on parsing the first data file received from the first healthcare provider computer system (e.g., healthcare provider computer system 120) to the one or more patient mobile devices (e.g., patient mobile device 150, patient mobile device 160), patient event management computing platform 110 may send a first notification to a first patient mobile device (e.g., patient mobile device 150), and the first notification may include a first link comprising a first unique token. In some instances, patient event management computing platform 110 may send such notifications via a text messaging service (e.g., SMS, MMS, or the like), via a push notification service, and/or via other messaging channels.

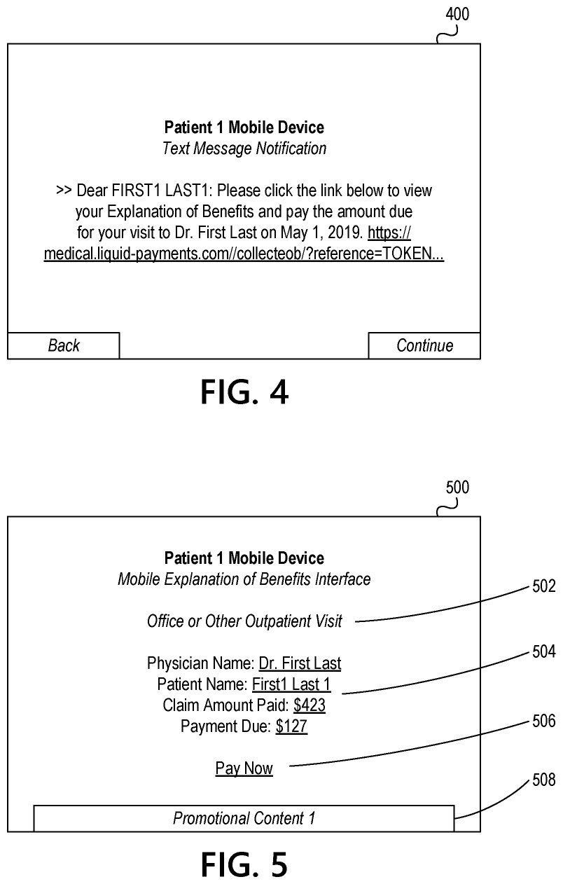

Referring to FIG. 2B, at step 205, patient mobile device 150 may receive a notification from patient event management computing platform 110. At step 206, patient mobile device 150 may present the notification received from patient event management computing platform 110. For example, in presenting the notification received from patient event management computing platform 110, patient mobile device 150 may display and/or otherwise present a graphical user interface similar to graphical user interface 400, which is illustrated in FIG. 4. As seen in FIG. 4, graphical user interface 400 may include text and/or other content associated with the notification generated by patient event management computing platform 110 (e.g., “Dear FIRST1 LAST1: Please click the link below to view your Explanation of Benefits and pay the amount due for your visit to Dr. First Last on May 1, 2019”) as well as a hyperlink with a unique token generated by patient event management computing platform 110 (e.g., “https://medical.liquid-payments.com//collecteob/?reference=TOKEN . . . ”), which may enable the user of patient mobile device 150 to access a mobile explanation-of-benefits user interface, as illustrated below.

At step 207, patient mobile device 150 may receive input selecting the link associated with the notification. For instance, patient mobile device 150 may receive such input from the user of patient mobile device 150. At step 208, patient mobile device 150 may send a request for the link to patient event management computing platform 110 (e.g., responsive to the link being selected and/or otherwise invoked by the user of patient mobile device 150).

Referring to FIG. 2C, at step 209, patient event management computing platform 110 may receive the request from patient mobile device 150. For example, at step 209, patient event management computing platform 110 may receive, via the communication interface (e.g., communication interface 113), from a first patient mobile device (e.g., patient mobile device 150), a first request for a first link comprising a first unique token. As discussed above, the unique token may have been previously generated by patient event management computing platform 110 and may be linked to a specific explanation-of-benefits record maintained by patient event management computing platform 110 (e.g., based on medical claim adjudication data received from one or more healthcare provider systems, such as healthcare provider computer system 120, healthcare provider computer system 130, and/or the like).

At step 210, patient event management computing platform 110 may load one or more patient-specific records associated with the token included in the request. For example, at step 210, responsive to receiving the first request for the first link comprising the first unique token, patient event management computing platform 110 may load one or more patient-specific records associated with the first link comprising the first unique token. For instance, patient event management computing platform 110 may load such records from patient event management database 112b (where, e.g., patient event management computing platform 110 may have previously stored such records based on medical claim adjudication data received from one or more healthcare provider systems, such as healthcare provider computer system 120, healthcare provider computer system 130, and/or the like).

At step 211, patient event management computing platform 110 may generate a mobile explanation-of-benefits interface based on the one or more records loaded at step 210. For example, at step 211, patient event management computing platform 110 may generate a first mobile explanation-of-benefits user interface based on the one or more patient-specific records associated with the first link comprising the first unique token. As illustrated in greater detail below, in generating such a user interface, patient event management computing platform 110 may manipulate and/or otherwise use data obtained from the one or more patient-specific records to insert content into the user interface being generated.

In some embodiments, generating the first mobile explanation-of-benefits user interface based on the one or more patient-specific records associated with the first link comprising the first unique token may include: identifying one or more current procedural terminology (CPT) codes as being present in the one or more patient-specific records associated with the first link comprising the first unique token; translating the one or more CPT codes identified as being present in the one or more patient-specific records into one or more visit descriptions; and inserting the one or more visit descriptions into the first mobile explanation-of-benefits user interface. For example, in generating the first mobile explanation-of-benefits user interface based on the one or more patient-specific records associated with the first link comprising the first unique token at step 211, patient event management computing platform 110 may identify one or more current procedural terminology (CPT) codes as being present in the one or more patient-specific records associated with the first link comprising the first unique token. Subsequently, patient event management computing platform 110 may translate the one or more CPT codes identified as being present in the one or more patient-specific records into one or more visit descriptions. For instance, different CPT codes may describe various medical services in a uniform fashion, and patient event management computing platform 110 may access a database storing information that links various CPT codes to corresponding visit descriptions, so as to translate a numerical CPT code into a plain-language, textual description of the services that were provided to a patient during a particular visit. Then, patient event management computing platform 110 may insert the one or more visit descriptions into the first mobile explanation-of-benefits user interface. As illustrated in greater detail below, by inserting the one or more visit descriptions into the first mobile explanation-of-benefits user interface, patient event management computing platform 110 may enable a patient to view and more easily understand the medical services for which a payment amount may be due.

In some embodiments, generating the first mobile explanation-of-benefits user interface based on the one or more patient-specific records associated with the first link comprising the first unique token may include inserting, based on the one or more patient-specific records, a physician name, a patient name, a claim-paid amount, and a payment-due amount into the first mobile explanation-of-benefits user interface. For example, in generating the first mobile explanation-of-benefits user interface based on the one or more patient-specific records associated with the first link comprising the first unique token at step 211, patient event management computing platform 110 may insert, based on the one or more patient-specific records, a physician name, a patient name, a claim-paid amount, and a payment-due amount into the first mobile explanation-of-benefits user interface.

In some embodiments, generating the first mobile explanation-of-benefits user interface based on the one or more patient-specific records associated with the first link comprising the first unique token may include inserting promotional content into the first mobile explanation-of-benefits user interface based on the one or more patient-specific records. For example, in generating the first mobile explanation-of-benefits user interface based on the one or more patient-specific records associated with the first link comprising the first unique token at step 211, patient event management computing platform 110 may insert promotional content into the first mobile explanation-of-benefits user interface based on the one or more patient-specific records. As illustrated below, such promotional content may be selected by patient event management computing platform 110 to be tailored to the recipient user based on contents of the one or more patient-specific records (and/or based on the recipient user having consented to receive such promotional content based on the contents of such records, for instance, in cases where such consent is required).

In some embodiments, inserting the promotional content into the first mobile explanation-of-benefits user interface based on the one or more patient-specific records may include selecting the promotional content from a library of available promotional content based on identifying one or more codes as being present in the one or more patient-specific records associated with the first link comprising the first unique token. For example, in inserting the promotional content into the first mobile explanation-of-benefits user interface based on the one or more patient-specific records, patient event management computing platform 110 may select the promotional content from a library of available promotional content (which may, e.g., be stored, maintained, and/or otherwise accessible to patient event management computing platform 110) based on identifying one or more codes as being present in the one or more patient-specific records associated with the first link comprising the first unique token. For instance, patient event management computing platform 110 may select promotional content associated with one or more specific products and/or services based on such products and/or services being related to health conditions and/or patient needs associated with specific CPT codes that may be present in the one or more patient-specific records associated with the first link comprising the first unique token. Additionally or alternatively, patient event management computing platform 110 may select promotional content based on other factors, such as patient location, device location, demographic information, and/or other information. For instance, for a given patient-user, patient event management computing platform 110 may select promotional content that advertises a specific medication (which may, e.g., be identified by patient event management computing platform 110 as being relevant to the patient-user based on a CPT code being included in a record associated with the patient-user) at a pharmacy that is local to the patient-user (which may, e.g., be identified by patient event management computing platform 110 as being proximate to the patient-user based on patient location and/or device location).

In some embodiments, inserting the promotional content into the first mobile explanation-of-benefits user interface based on the one or more patient-specific records may include selecting the promotional content from a library of available promotional content based on identifying a physician name included in the one or more patient-specific records associated with the first link comprising the first unique token. For example, in inserting the promotional content into the first mobile explanation-of-benefits user interface based on the one or more patient-specific records, patient event management computing platform 110 may select the promotional content from a library of available promotional content based on identifying a physician name included in the one or more patient-specific records associated with the first link comprising the first unique token. For instance, patient event management computing platform 110 may select promotional content associated with one or more specific products and/or services based on such products and/or services being related to health conditions and/or patient needs associated with a specialty of a physician seen by the patient-user (which may, e.g., be identified by patient event management computing platform 110 based on the one or more patient-specific records associated with the first link comprising the first unique token identifying the name of such a physician). Additionally or alternatively, patient event management computing platform 110 may select promotional content based on other factors, such as patient location, device location, demographic information, and/or other information. For instance, for a given patient-user, patient event management computing platform 110 may select promotional content that advertises a specific follow-up clinic or practice (which may, e.g., be identified by patient event management computing platform 110 as being relevant to the patient-user based on a physician name being included in a record associated with the patient-user) at an office that is local to the patient-user (which may, e.g., be identified by patient event management computing platform 110 as being proximate to the patient-user based on patient location and/or device location).

At step 212, patient event management computing platform 110 may send the mobile explanation-of-benefits interface (which may, e.g., have been generated by patient event management computing platform 110 at step 211) to patient mobile device 150. For example, at step 212, patient event management computing platform 110 may send, via the communication interface (e.g., communication interface 113), to the first patient mobile device (e.g., patient mobile device 150), the first mobile explanation-of-benefits user interface generated based on the one or more patient-specific records associated with the first link comprising the first unique token. In sending the first mobile explanation-of-benefits user interface to the first patient mobile device (e.g., patient mobile device 150), patient event management computing platform 110 may cause the first patient mobile device (e.g., patient mobile device 150) to display and/or otherwise present a graphical user interface similar to graphical user interface 500, which is illustrated in FIG. 5. As seen in FIG. 5, graphical user interface 500 may include visit description text 502 (which may, e.g., have been translated by patient event management computing platform 110 based on a CPT code included in a patient record), detailed explanation-of-benefits information 504 (which may, e.g., be obtained from and/or determined from the patient record and may include a physician name, a patient name, a claim amount paid value, and a payment due value), a pay-now button 506 (which may, e.g., be user-selectable to enable a user of patient mobile device 150 to pay the amount due), and promotional content 508 (which may, e.g., be selected and/or inserted by patient event management computing platform 110, as described above). In some instances, a particular patient may have multiple different records with multiple different CPT codes, in which case additional visit description text and explanation-of-benefits information may be included in the mobile explanation-of-benefits user interface that is generated by patient event management computing platform 110 and presented by patient mobile device 150.

Referring to FIG. 2D, at step 213, patient mobile device 150 may present the mobile explanation-of-benefits user interface. For example, patient mobile device 150 may display and/or otherwise present the mobile explanation-of-benefits user interface received from patient event management computing platform 110. In addition, patient mobile device 150 may receive user input via the mobile explanation-of-benefits user interface (which may, e.g., include a selection of the pay-now button 506, which may trigger patient mobile device 150 to send a request to submit a payment to patient event management computing platform 110).

At step 214, patient event management computing platform 110 may receive a request to submit a payment from patient mobile device 150. For example, at step 214, patient event management computing platform 110 may receive, via the communication interface (e.g., communication interface 113), from the first patient mobile device (e.g., patient mobile device 150), a request to submit a payment associated with a payment-due amount included in the first mobile explanation-of-benefits user interface. For instance, such a request may be initiated by the user of patient mobile device 150 via the first mobile explanation-of-benefits user interface.

At step 215, patient event management computing platform 110 may determine whether saved payment method information is available for the patient-user associated with patient mobile device 150. For example, at step 215, responsive to receiving the request to submit the payment associated with the payment-due amount included in the first mobile explanation-of-benefits user interface, patient event management computing platform 110 may determine whether saved payment method information linked to a user of the first patient mobile device (e.g., patient mobile device 150) is available. To determine whether saved payment method information linked to a user of the first patient mobile device (e.g., patient mobile device 150) is available, patient event management computing platform 110 may access records stored in a secure database (e.g., patient event management database 112b) in which patient event management computing platform 110 may store and/or otherwise maintain payment information for different patient-users.

At step 216, if saved payment method information is available for the patient-user associated with patient mobile device 150, patient event management computing platform 110 may process the requested payment. For example, at step 216, based on determining that the saved payment method information linked to the user of the first patient mobile device (e.g., patient mobile device 150) is available, patient event management computing platform 110 may process the payment associated with the payment-due amount included in the first mobile explanation-of-benefits user interface using the saved payment method information linked to the user of the first patient mobile device (e.g., patient mobile device 150). For instance, the saved payment method information linked to the user of the first patient mobile device (e.g., patient mobile device 150) may include a saved credit card number or debit card number, an expiration date, a security code, and a cardholder name, and patient event management computing platform 110 may process the payment by directing and/or controlling a payment server (e.g., remote event processing server 140) to complete a payment transaction using the saved payment method information. For example, patient event management computing platform 110 may generate and send one or more formatted commands directing remote event processing server 140 to complete a payment event corresponding to the requested payment.

Referring to FIG. 2E, at step 217, if saved payment method information is not available for the patient-user associated with patient mobile device 150, patient event management computing platform 110 may generate and send one or more user interfaces that are configured to prompt the patient-user to provide payment method information that can be used to process the requested payment. For example, at step 217, based on determining that the saved payment method information linked to the user of the first patient mobile device (e.g., patient mobile device 150) is not available, patient event management computing platform 110 may generate a first payment-details user interface based on the one or more patient-specific records associated with the first link comprising the first unique token.

In some embodiments, generating the first payment-details user interface based on the one or more patient-specific records associated with the first link comprising the first unique token may include inserting promotional content into the first payment-details user interface based on the one or more patient-specific records. For example, in generating the first payment-details user interface based on the one or more patient-specific records associated with the first link comprising the first unique token at step 217, patient event management computing platform 110 may insert promotional content into the first payment-details user interface based on the one or more patient-specific records. For instance, patient event management computing platform 110 may select and insert a targeted advertisement into the payment-details user interface, similar to how patient event management computing platform 110 may select and insert a targeted advertisement into the mobile explanation-of-benefits user interface, as described above. The promotional content that is inserted into the payment-details user interface by patient event management computing platform 110 may, for instance, be different from the promotional content that is inserted into the mobile explanation-of-benefits user interface. Additionally or alternatively, the promotional content that is inserted into the payment-details user interface by patient event management computing platform 110 may, for instance, be selected and inserted by patient event management computing platform 110 from a library of available promotional content (e.g., based on identifying one or more codes, physician names, location data, demographic data, and/or other data as being present in the one or more patient-specific records associated with the first link comprising the first unique token, similar to the examples described above in which promotional content may be selected and inserted by patient event management computing platform 110 into the mobile explanation-of-benefits user interface).

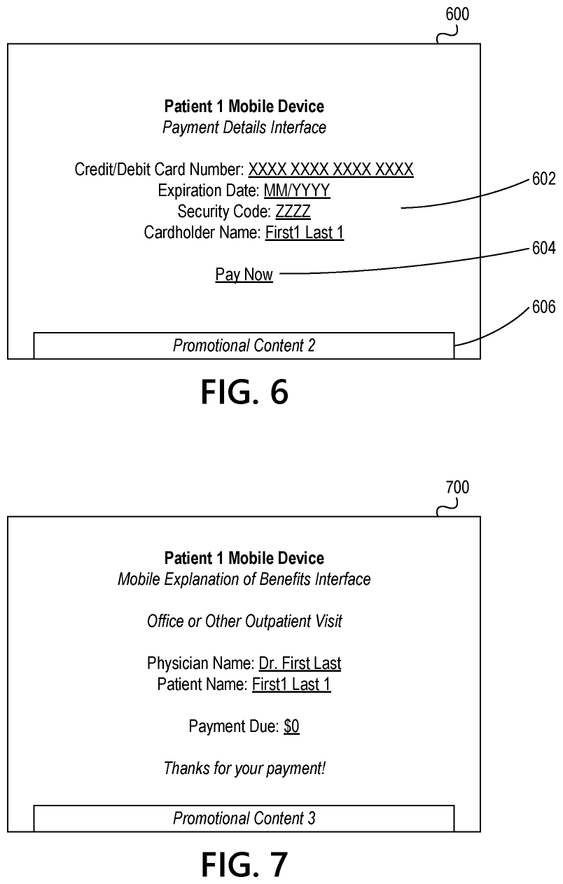

At step 218, patient event management computing platform 110 may send the payment-details user interface to patient mobile device 150. For example, at step 218, patient event management computing platform 110 may send, via the communication interface (e.g., communication interface 113), to the first patient mobile device (e.g., patient mobile device 150), the first payment-details user interface generated based on the one or more patient-specific records associated with the first link comprising the first unique token. In sending the first payment-details user interface to the first patient mobile device (e.g., patient mobile device 150), patient event management computing platform 110 may cause the first patient mobile device (e.g., patient mobile device 150) to display and/or otherwise present a graphical user interface similar to graphical user interface 600, which is illustrated in FIG. 6. As seen in FIG. 6, graphical user interface 600 may include one or more fields 602 in which a user enter a credit/debit card number, an expiration date, a security code, and a cardholder name. Graphical user interface 600 also may include a pay-now button 604, as well as promotional content 606 (which may, e.g., be selected and inserted by patient event management computing platform 110, as described above).

At step 219, patient mobile device 150 may display and/or otherwise present the payment-details user interface received from patient event management computing platform 110. In addition, patient mobile device 150 may receive user input via the payment-details user interface (which may, e.g., include entry of new payment information via fields 602 and/or a selection of the pay-now button 604).

At step 220, patient event management computing platform 110 may receive new payment information from patient mobile device 150. For example, at step 220, after sending the first payment-details user interface to the first patient mobile device (e.g., patient mobile device 150), patient event management computing platform 110 may receive, via the communication interface (e.g., communication interface 113), from the first patient mobile device (e.g., patient mobile device 150), new payment information linked to the user of the first patient mobile device (e.g., patient mobile device 150).

Referring to FIG. 2F, at step 221, patient event management computing platform 110 may store the new payment information received from patient mobile device 150. For example, at step 221, patient event management computing platform 110 may store the new payment information linked to the user of the first patient mobile device (e.g., patient mobile device 150) received from the first patient mobile device (e.g., patient mobile device 150). For instance, patient event management computing platform 110 may securely store such payment information in a PCI compliant database, such as patient event management database 112b.

At step 222, patient event management computing platform 110 may process the requested payment for the patient-user associated with patient mobile device 150. For example, at step 222, after storing the new payment information linked to the user of the first patient mobile device (e.g., patient mobile device 150) received from the first patient mobile device (e.g., patient mobile device 150), patient event management computing platform 110 may process the payment associated with the payment-due amount included in the first mobile explanation-of-benefits user interface using the new payment information linked to the user of the first patient mobile device (e.g., patient mobile device 150). For instance, patient event management computing platform 110 may process the payment by directing and/or controlling a payment server (e.g., remote event processing server 140) to complete a payment transaction using the credit/debit card number, expiration date, security code, and cardholder name included in the new payment information linked to the user of the first patient mobile device (e.g., patient mobile device 150). For example, patient event management computing platform 110 may generate and send one or more formatted commands directing remote event processing server 140 to complete a payment event corresponding to the requested payment.

At step 223, patient event management computing platform 110 may generate an updated mobile explanation-of-benefits user interface (e.g., based on completing and/or otherwise processing the requested payment). For example, at step 223, after processing the payment associated with the payment-due amount included in the first mobile explanation-of-benefits user interface, patient event management computing platform 110 may generate a second mobile explanation-of-benefits user interface for the user of the first patient mobile device (e.g., patient mobile device 150). This updated interface (which may, e.g., be generated by patient event management computing platform 110) may, for instance, indicate an updated amount due (e.g., a zero balance), as well as the patient-user's visit history and/or payment history (which may, e.g., include explanation-of-benefits information and/or payment history information associated with other, previous physician visits by the patient-user, in addition to the patient-user's most recent visit and corresponding explanation of benefits).

At step 224, patient event management computing platform 110 may send the updated mobile explanation-of-benefits user interface to patient mobile device 150. For example, at step 224, patient event management computing platform 110 may send, via the communication interface (e.g., communication interface 113), to the first patient mobile device (e.g., patient mobile device 150), the second mobile explanation-of-benefits user interface generated for the user of the first patient mobile device (e.g., patient mobile device 150). In some instances, in sending the second mobile explanation-of-benefits user interface to the first patient mobile device (e.g., patient mobile device 150), patient event management computing platform 110 may cause the first patient mobile device (e.g., patient mobile device 150) to display and/or otherwise present a graphical user interface similar to graphical user interface 700, which is illustrated in FIG. 7. As seen in FIG. 7, graphical user interface 700 may include updated explanation-of-benefits information indicating that the previously-requested payment has been processed and that the patient-user has a zero balance.

Referring to FIG. 2G, at step 225, patient mobile device 150 may display and/or otherwise present the updated mobile explanation-of-benefits user interface received from patient event management computing platform 110. At step 226, patient mobile device 160 may receive input selecting a second link associated with a second notification (which may, e.g., have been sent by patient event management computing platform 110 to patient mobile device 160 at step 204). For instance, patient mobile device 160 may receive such input from the user of patient mobile device 160. At step 227, patient mobile device 160 may send a request for the second link to patient event management computing platform 110 (e.g., responsive to the link being selected and/or otherwise invoked by the user of patient mobile device 160).

At step 228, patient event management computing platform 110 may receive the request from patient mobile device 160. For example, at step 228, patient event management computing platform 110 may receive, via the communication interface (e.g., communication interface 113), from a second patient mobile device (e.g., patient mobile device 160), a second request for a second link comprising a second unique token. As discussed above, the unique token may have been previously generated by patient event management computing platform 110 and may be linked to a specific explanation-of-benefits record maintained by patient event management computing platform 110 (e.g., based on medical claim adjudication data received from one or more healthcare provider systems, such as healthcare provider computer system 120, healthcare provider computer system 130, and/or the like).

Referring to FIG. 2H, at step 229, patient event management computing platform 110 may load one or more patient-specific records associated with the token included in the request. For example, at step 229, responsive to receiving the second request for the second link comprising the second unique token, patient event management computing platform 110 may load one or more patient-specific records associated with the second link comprising the second unique token. For instance, patient event management computing platform 110 may load such records from patient event management database 112b (where, e.g., patient event management computing platform 110 may have previously stored such records based on medical claim adjudication data received from one or more healthcare provider systems, such as healthcare provider computer system 120, healthcare provider computer system 130, and/or the like).

At step 230, patient event management computing platform 110 may generate a mobile explanation-of-benefits interface based on the one or more records loaded at step 229. For example, at step 230, patient event management computing platform 110 may generate a third mobile explanation-of-benefits user interface based on the one or more patient-specific records associated with the second link comprising the second unique token. For instance, patient event management computing platform 110 may generate such a mobile explanation-of-benefits user interface similar to how patient event management computing platform 110 generated the first mobile explanation-of-benefits user interface, as described above. In addition, and like in the examples discussed above, in generating such a user interface, patient event management computing platform 110 may manipulate and/or otherwise use data obtained from the one or more patient-specific records to insert content into the user interface.

At step 231, patient event management computing platform 110 may send the mobile explanation-of-benefits interface to patient mobile device 160. For example, at step 231, patient event management computing platform 110 may send, via the communication interface (e.g., communication interface 113), to the second patient mobile device (e.g., patient mobile device 160), the third mobile explanation-of-benefits user interface generated based on the one or more patient-specific records associated with the second link comprising the second unique token. In sending the third mobile explanation-of-benefits user interface to the second patient mobile device (e.g., patient mobile device 160), patient event management computing platform 110 may cause the second patient mobile device (e.g., patient mobile device 160) to display and/or otherwise present one or more graphical user interfaces, like in the examples discussed above.

At step 232, patient event management computing platform 110 may process a payment requested by the patient-user of patient mobile device 160. For example, at step 232, patient event management computing platform 110 may process a payment based on one or more requests received from patient mobile device 160, stored payment information linked to patient mobile device 160, and/or new payment information received from patient mobile device 160 (e.g., similar to how patient event management computing platform 110 may process a payment requested from patient mobile device 150 in the examples discussed above).

Subsequently, patient event management computing platform 110 may repeat one or more processing steps similar to those described above. For example, patient event management computing platform 110 may receive additional data files with new explanations-of-benefits information from healthcare provider computer system 120, healthcare provider computer system 130, and/or other systems and/or devices. In addition, patient event management computing platform 110 may generate and/or send notifications to patient mobile device 150, patient mobile device 160, and/or other patient-user devices, and may process requests and/or payments associated with such user devices, similar to how patient event management computing platform 110 may process the requests and payments described above. By performing one or more of the processing steps described above, patient event management computing platform 110 may provide an efficient and convenient user experience to both doctors and patients (e.g., by enabling patients to securely and easily make payments from their personal mobile device, rather than requiring them to use other, cumbersome tools).

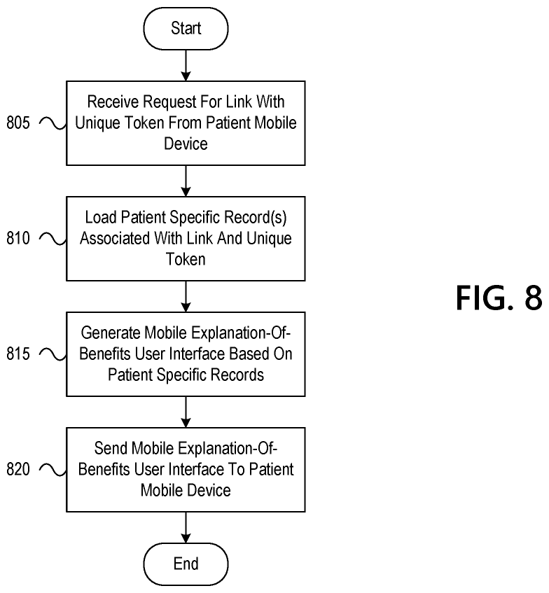

FIG. 8 depicts an illustrative method for dynamically generating patient-facing mobile interfaces based on healthcare data retrieved from secure medical information systems in accordance with one or more example embodiments. Referring to FIG. 8, at step 805, a computing platform having at least one processor, a communication interface, and memory may receive, via the communication interface, from a first patient mobile device, a first request for a first link comprising a first unique token. At step 810, responsive to receiving the first request for the first link comprising the first unique token, the computing platform may load one or more patient-specific records associated with the first link comprising the first unique token. At step 815, the computing platform may generate a first mobile explanation-of-benefits user interface based on the one or more patient-specific records associated with the first link comprising the first unique token. At step 820, the computing platform may send, via the communication interface, to the first patient mobile device, the first mobile explanation-of-benefits user interface generated based on the one or more patient-specific records associated with the first link comprising the first unique token.

One or more aspects of the disclosure may be embodied in computer-usable data or computer-executable instructions, such as in one or more program modules, executed by one or more computers or other devices to perform the operations described herein. Program modules may include routines, programs, objects, components, data structures, and the like that perform particular tasks or implement particular data types when executed by one or more processors in a computer or other data processing device. The computer-executable instructions may be stored as computer-readable instructions on a computer-readable medium such as a hard disk, optical disk, removable storage media, solid-state memory, RAM, and the like. The functionality of the program modules may be combined or distributed as desired in various embodiments. In addition, the functionality may be embodied in whole or in part in firmware or hardware equivalents, such as integrated circuits, application-specific integrated circuits (ASICs), field programmable gate arrays (FPGA), and the like. Particular data structures may be used to more effectively implement one or more aspects of the disclosure, and such data structures are contemplated to be within the scope of computer executable instructions and computer-usable data described herein.

Various aspects described herein may be embodied as a method, an apparatus, or as one or more computer-readable media storing computer-executable instructions. Accordingly, those aspects may take the form of an entirely hardware embodiment, an entirely software embodiment, an entirely firmware embodiment, or an embodiment combining software, hardware, and firmware aspects in any combination. In addition, various signals representing data or events as described herein may be transferred between a source and a destination in the form of light or electromagnetic waves traveling through signal-conducting media such as metal wires, optical fibers, or wireless transmission media. The one or more computer-readable media may be and/or include one or more non-transitory computer-readable media.

As described herein, the various methods and acts may be operative across one or more computing servers and one or more networks. The functionality may be distributed in any manner, or may be located in a single computing device (e.g., a server, a client computer, and the like). In some instances, one or more of the computing platforms discussed above may be implemented in one or more virtual machines that are provided by one or more physical computing devices. In such arrangements, the various functions of each computing platform may be performed by the one or more virtual machines, and any and/or all of the above-discussed communications between computing platforms may correspond to data being accessed, moved, modified, updated, and/or otherwise used by the one or more virtual machines.

Aspects of the disclosure have been described in terms of illustrative embodiments thereof. Numerous other embodiments, modifications, and variations within the scope and spirit of the appended claims will occur to persons of ordinary skill in the art from a review of this disclosure. For example, one or more of the steps depicted in the illustrative figures may be performed in other than the recited order, and one or more depicted steps may be optional in accordance with aspects of the disclosure.

What is claimed is:

1. A computing platform, comprising:
at least one processor;
a communication interface communicatively coupled to the at least one processor; and
memory storing computer-readable instructions that, when executed by the at least one processor, cause the computing platform to:
receive, via the communication interface, from a first patient mobile device, a first request for a first link comprising a first unique token;
responsive to receiving the first request for the first link comprising the first unique token, load one or more patient-specific records associated with the first link comprising the first unique token;
generate a first mobile explanation-of-benefits user interface based on the one or more patient-specific records associated with the first link comprising the first unique token; and
send, via the communication interface, to the first patient mobile device, the first mobile explanation-of-benefits user interface generated based on the one or more patient-specific records associated with the first link comprising the first unique token.

2. The computing platform of claim 1, wherein the memory stores additional computer-readable instructions that, when executed by the at least one processor, cause the computing platform to:
prior to receiving the first request for the first link comprising the first unique token:
receive, via the communication interface, from a first healthcare provider computer system, a first data file comprising at least one patient-specific record associated with at least one patient;
parse the first data file received from the first healthcare provider computer system;
based on parsing the first data file received from the first healthcare provider computer system, generate one or more patient-specific notifications; and
send, via the communication interface, to one or more patient mobile devices, the one or more patient-specific notifications generated based on parsing the first data file received from the first healthcare provider computer system,
wherein sending the one or more patient-specific notifications generated based on parsing the first data file received from the first healthcare provider computer system to the one or more patient mobile devices comprises sending a first notification to the first patient mobile device, and wherein the first notification comprises the first link comprising the first unique token.

3. The computing platform of claim 2,
wherein the first healthcare provider computer system comprises a secure file transfer protocol (SFTP) server, and
wherein receiving the first data file comprising the at least one patient-specific record associated with the at least one patient from the first healthcare provider computer system comprises:
establishing a secure connection with the SFTP server; and
downloading the first data file comprising the at least one patient-specific record associated with the at least one patient via the secure connection with the SFTP server.

4. The computing platform of claim 1, wherein generating the first mobile explanation-of-benefits user interface based on the one or more patient-specific records associated with the first link comprising the first unique token comprises:
identifying one or more current procedural terminology (CPT) codes as being present in the one or more patient-specific records associated with the first link comprising the first unique token;
translating the one or more CPT codes identified as being present in the one or more patient-specific records into one or more visit descriptions; and
inserting the one or more visit descriptions into the first mobile explanation-of-benefits user interface.

5. The computing platform of claim 1, wherein generating the first mobile explanation-of-benefits user interface based on the one or more patient-specific records associated with the first link comprising the first unique token comprises inserting, based on the one or more patient-specific records, a physician name, a patient name, a claim-paid amount, and a payment-due amount into the first mobile explanation-of-benefits user interface.

6. The computing platform of claim 1, wherein generating the first mobile explanation-of-benefits user interface based on the one or more patient-specific records associated with the first link comprising the first unique token comprises inserting promotional content into the first mobile explanation-of-benefits user interface based on the one or more patient-specific records.

7. The computing platform of claim 6, wherein inserting the promotional content into the first mobile explanation-of-benefits user interface based on the one or more patient-specific records comprises selecting the promotional content from a library of available promotional content based on identifying one or more codes as being present in the one or more patient-specific records associated with the first link comprising the first unique token.

8. The computing platform of claim 6, wherein inserting the promotional content into the first mobile explanation-of-benefits user interface based on the one or more patient-specific records comprises selecting the promotional content from a library of available promotional content based on identifying a physician name included in the one or more patient-specific records associated with the first link comprising the first unique token.

9. The computing platform of claim 1, wherein the memory stores additional computer-readable instructions that, when executed by the at least one processor, cause the computing platform to:
receive, via the communication interface, from the first patient mobile device, a request to submit a payment associated with a payment-due amount included in the first mobile explanation-of-benefits user interface;
responsive to receiving the request to submit the payment associated with the payment-due amount included in the first mobile explanation-of-benefits user interface, determine whether saved payment method information linked to a user of the first patient mobile device is available; and
based on determining that the saved payment method information linked to the user of the first patient mobile device is available, process the payment associated with the payment-due amount included in the first mobile explanation-of-benefits user interface using the saved payment method information linked to the user of the first patient mobile device.

10. The computing platform of claim 9, wherein the memory stores additional computer-readable instructions that, when executed by the at least one processor, cause the computing platform to:
based on determining that the saved payment method information linked to the user of the first patient mobile device is not available:
generate a first payment-details user interface based on the one or more patient-specific records associated with the first link comprising the first unique token; and
send, via the communication interface, to the first patient mobile device, the first payment-details user interface generated based on the one or more patient-specific records associated with the first link comprising the first unique token.

11. The computing platform of claim 10, wherein generating the first payment-details user interface based on the one or more patient-specific records associated with the first link comprising the first unique token comprises inserting promotional content into the first payment-details user interface based on the one or more patient-specific records.

12. The computing platform of claim 10, wherein the memory stores additional computer-readable instructions that, when executed by the at least one processor, cause the computing platform to:
after sending the first payment-details user interface to the first patient mobile device, receive, via the communication interface, from the first patient mobile device, new payment information linked to the user of the first patient mobile device; and
store the new payment information linked to the user of the first patient mobile device received from the first patient mobile device.

13. The computing platform of claim 12, wherein the memory stores additional computer-readable instructions that, when executed by the at least one processor, cause the computing platform to:
after storing the new payment information linked to the user of the first patient mobile device received from the first patient mobile device, process the payment associated with the payment-due amount included in the first mobile explanation-of-benefits user interface using the new payment information linked to the user of the first patient mobile device.

14. The computing platform of claim 9, wherein the memory stores additional computer-readable instructions that, when executed by the at least one processor, cause the computing platform to:
after processing the payment associated with the payment-due amount included in the first mobile explanation-of-benefits user interface, generate a second mobile explanation-of-benefits user interface for the user of the first patient mobile device; and
send, via the communication interface, to the first patient mobile device, the second mobile explanation-of-benefits user interface generated for the user of the first patient mobile device.

15. The computing platform of claim 14, wherein the memory stores additional computer-readable instructions that, when executed by the at least one processor, cause the computing platform to:
receive, via the communication interface, from a second patient mobile device, a second request for a second link comprising a second unique token;
responsive to receiving the second request for the second link comprising the second unique token, load one or more patient-specific records associated with the second link comprising the second unique token;
generate a third mobile explanation-of-benefits user interface based on the one or more patient-specific records associated with the second link comprising the second unique token; and
send, via the communication interface, to the second patient mobile device, the third mobile explanation-of-benefits user interface generated based on the one or more patient-specific records associated with the second link comprising the second unique token.

16. A method, comprising:
at a computing platform comprising at least one processor, memory, and a communication interface:
receiving, by the at least one processor, via the communication interface, from a first patient mobile device, a first request for a first link comprising a first unique token;
responsive to receiving the first request for the first link comprising the first unique token, loading, by the at least one processor, one or more patient-specific records associated with the first link comprising the first unique token;
generating, by the at least one processor, a first mobile explanation-of-benefits user interface based on the one or more patient-specific records associated with the first link comprising the first unique token; and
sending, by the at least one processor, via the communication interface, to the first patient mobile device, the first mobile explanation-of-benefits user interface generated based on the one or more patient-specific records associated with the first link comprising the first unique token.

17. The method of claim 16, comprising:
prior to receiving the first request for the first link comprising the first unique token:
receiving, by the at least one processor, via the communication interface, from a first healthcare provider computer system, a first data file comprising at least one patient-specific record associated with at least one patient;
parsing, by the at least one processor, the first data file received from the first healthcare provider computer system;
based on parsing the first data file received from the first healthcare provider computer system, generating, by the at least one processor, one or more patient-specific notifications; and
sending, by the at least one processor, via the communication interface, to one or more patient mobile devices, the one or more patient-specific notifications generated based on parsing the first data file received from the first healthcare provider computer system,
wherein sending the one or more patient-specific notifications generated based on parsing the first data file received from the first healthcare provider computer system to the one or more patient mobile devices comprises sending a first notification to the first patient mobile device, and wherein the first notification comprises the first link comprising the first unique token.

18. The method of claim 17,
wherein the first healthcare provider computer system comprises a secure file transfer protocol (SFTP) server, and
wherein receiving the first data file comprising the at least one patient-specific record associated with the at least one patient from the first healthcare provider computer system comprises:
establishing a secure connection with the SFTP server; and
downloading the first data file comprising the at least one patient-specific record associated with the at least one patient via the secure connection with the SFTP server.

19. The method of claim 16, wherein generating the first mobile explanation-of-benefits user interface based on the one or more patient-specific records associated with the first link comprising the first unique token comprises:
identifying one or more current procedural terminology (CPT) codes as being present in the one or more patient-specific records associated with the first link comprising the first unique token;
translating the one or more CPT codes identified as being present in the one or more patient-specific records into one or more visit descriptions; and
inserting the one or more visit descriptions into the first mobile explanation-of-benefits user interface.

20. One or more non-transitory computer-readable media storing instructions that, when executed by a computing platform comprising at least one processor, memory, and a communication interface, cause the computing platform to:
receive, via the communication interface, from a first patient mobile device, a first request for a first link comprising a first unique token;
responsive to receiving the first request for the first link comprising the first unique token, load one or more patient-specific records associated with the first link comprising the first unique token;
generate a first mobile explanation-of-benefits user interface based on the one or more patient-specific records associated with the first link comprising the first unique token; and
send, via the communication interface, to the first patient mobile device, the first mobile explanation-of-benefits user interface generated based on the one or more patient-specific records associated with the first link comprising the first unique token.
